SEQUENCE LISTING (22 sequences)

SEQ ID NO: 1 - protein, 275 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDSNSNDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 313 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISESSFAQPMNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 3 - DNA, 828 bp, Homo sapiens
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagactcaaattctaacgactacaagctcagctttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 4 - DNA, 942 bp, Homo sapiens
atgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagtcgagcttcgctcagcccatgaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 5 - protein, 114 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRD

SEQ ID NO: 6 - DNA, 342 bp, Homo sapiens
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagac

SEQ ID NO: 7 - protein, 115 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISES

SEQ ID NO: 8 - DNA, 345 bp, Homo sapiens
atgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagtcg

SEQ ID NO: 9 - protein, 9 aa, Homo sapiens
YLILEYAPL

SEQ ID NO: 10 - DNA, 40 bp, Artificial Sequence
  Oligo dT adaptor
ctgtgaatgctgcgactacgattttttttttttttttttt

SEQ ID NO: 11 - DNA, 22 bp, Artificial Sequence
  5′-RACE primer
gtctaccaggcattcgcttcat

SEQ ID NO: 12 - DNA, 24 bp, Artificial Sequence
  Synthetic primer 3-TRalpha-C to amplify a DNA fragment encoding TCR alpha chain
tcagctggaccacagccgcagcgt

SEQ ID NO: 13 - DNA, 21 bp, Artificial Sequence
  Synthetic primer 3-TRbeta-C1 to amplify a DNA fragment encoding TCR beta chain
tcagaaatcctttctcttgac

SEQ ID NO: 14 - DNA, 24 bp, Artificial Sequence
  Synthetic primer 3-TRbeta-C2 to amplify a DNA fragment encoding TCR beta chain
ctagcctctggaatcctttctctt

SEQ ID NO: 15 - DNA, 34 bp, Artificial Sequence
  Synthetic primer
tacctcgagcgataaaataaaagattttatttag

SEQ ID NO: 16 - DNA, 45 bp, Artificial Sequence
  Synthetic primer
tacgaattcgattgaatccgtcgactgaaagacccccgctgacgg

SEQ ID NO: 17 - DNA, 40 bp, Artificial Sequence
  Synthetic primer
atttatttgcggccgccaccatggcctctgcacccatctc

SEQ ID NO: 18 - DNA, 31 bp, Artificial Sequence
  Synthetic primer
aataaggatcctcagctggaccacagccgca

SEQ ID NO: 19 - DNA, 37 bp, Artificial Sequence
  Synthetic primer
atttagcggccgccaccatgggcacaaggttgttctt

SEQ ID NO: 20 - DNA, 29 bp, Artificial Sequence
  Synthetic primer
gggggatcctcagaaatcctttctcttga

SEQ ID NO: 21 - DNA, 39 bp, Artificial Sequence
  Synthetic primer
acgcgtgcggccgcagatctaattctaccgggtagggga

SEQ ID NO: 22 - DNA, 40 bp, Artificial Sequence
  Synthetic primer
ctcgaggcggccgcggatcccaggtcgaaaggcccggaga

(19) United States
(12) Patent Application Publication
(10) Pub. No.: US 2012/0009162 A1
(43) Pub. Date: Jan. 12, 2012

(54) T CELL RECEPTOR AND NUCLEIC ACID ENCODING THE RECEPTOR

(76) Inventors: Masaki Yasukawa, Toon-shi (JP); Hiroshi Fujiwara, Toon-shi (JP); Toshiki Ochi, Toon-shi (JP); Junichi Mineno, Otsu-shi (JP); Ikunoshin Kato, Otsu-shi (JP)

(21) Appl. No.: 13/257,389

(22) PCT Filed: Apr. 2, 2010
(86) PCT No.: PCT/JP2010/056089

§ 371 Date: Sep. 19, 2011

(30) Foreign Application Priority Data
Apr. 3, 2009 (JP) 2009-091384

(51) Int. Cl. A61K 35/12; C12N 15/63; A61P 35/00; C07K 14/435; C07H 21/04; C12N 5/10; A61K 31/7088

(52) U.S. Cl. 424/93.21; 435/325; 435/320.1; 514/44 R; 530/350; 536/23.5

(57) ABSTRACT

Disclosed are: polypeptides for TCR α-chain and β-chain which are restricted to HLA-A*0201 for Aur-A and are derived from a CTL; and nucleic acids encoding the polypeptides. The nucleic acids can impart a cytotoxic activity against a cell capable of presenting an HLA-A*0201 molecule and an Aur-A₂₀₇₋₂₁₅ peptide thereon to a T cell, and are therefore useful for the treatment of cancer in which Aur-A is expressed.

### TECHNICAL FIELD

The present invention relates to polypeptides composing an Aur-A₂₀₇₋₂₁₅ peptide-specific and HLA-A*0201-restricted T cell receptor (TCR), nucleic acids encoding the polypeptides, a T cell receptor composed of the polypeptides, a recombinant nucleic acid comprising the nucleic acid, a vector comprising the recombinant nucleic acid, a cell in which the nucleic acid or the vector is introduced, and a carcinostatic agent comprising the vector or the cell as an active ingredient.

### BACKGROUND ART

Among cytotoxic T cells (CTLs), some CTLs can recognize a complex formed by binding of a major histocompatibility antigen molecule (MHC molecule, referred to as a human leukocyte antigen in the case of human; hereinafter, abbreviated as HLA) encoded by a major histocompatibility gene complex (hereinafter, abbreviated as MHC) and an antigen peptide via a specific T cell receptor (hereinafter, abbreviated as TCR) to damage a cell presenting the complex on the cell surface. Therefore, in order to achieve the cytotoxic response, 1) the existence of a CTL having a TCR specific for an HLA class I type of a target cell and 2) the existence of an antigen peptide wherein a TCR can recognize a complex formed by binding of said antigen peptide and an HLA molecule are required.

Such an antigen peptide is generated, for example, by degradation of an antigen, etc. synthesized in a mammal cell to a peptide of 8 to 15 amino acids by processing in a cytoplasm. The peptide is then associated with an HLA molecule, and is presented on the cell surface.

The HLA class I molecule is roughly classified into subtypes of HLA-A, -B and -C. It is known that an antigen peptide which binds to the HLA molecule and then is presented on the cell surface consists of 8 to 10 amino acids. Further, it is known that the antigen peptide has a certain structural feature which is different depending on each HLA molecule. For example, as a peptide capable of binding to an HLA-A*0201 molecule which the largest number of people in the world have, a peptide consisting of 9 to 10 amino acids wherein Leu is at the second position from the N-terminal and Leu or Val is at the C-terminal is known. In addition, as a peptide capable of binding to an HLA-A24 molecule which many Asian races including Japanese have, there is a peptide consisting of 9 to 10 amino acids wherein Tyr, Phe, Net or Trp is at the second position from the N-terminal and Leu, Ile, Trp or Phe is at the C-terminal.

Examples of tumor antigens whose antigenic peptides have been identified include MAGE-A1, MAGE-A2, MAGE-A3, MAGE-A4, MART-1, tyrosinase, gp100, HER2/neu, CEA, NY-ESO-1, and β-catenin. Peptides derived from these antigens, each peptide of which binds to one or plural kinds of subtypes of HLA molecules are known. Many of the peptides have been found by establishing a strain of class I-restricted CTLs capable of recognizing tumor cells, identifying a tumor antigen recognized by the CTL, and subsequently determining a minimum unit peptide in the tumor antigen protein by a genetic engineering method referring to information about motifs binding to HLA class I molecules. In addition, the antigenic peptide has been also determined by finding out HLA class I molecule-binding peptide candidates in the tumor antigen protein based on a motif structure common to HLA class I molecule-binding peptides, and subsequently selecting a peptide which can induce a CTL having cytotoxicity on a tumor cell utilizing an antigen-presenting cell and the candidate peptide.

The distribution of subtypes of HLA class I molecules greatly varies between races. Worldwide, HLA-A02 accounts for the greatest proportion and 45% of Caucasians are HLA-A02-positive. In the case of Japanese, HLA-A02 positive people account for 40%. Of Japanese with said subtype, 20% are HLA-A*0201 positive like Caucasians, and many others are A*0206 positive. Therefore, discovery of an HLA-A02-restricted antigen peptide plays an important role in providing a CTL useful for tumor treatment by inducing a CTL which specifically acts on tumor cells.

Antigen peptides are different depending on different HLAs, even if they are derived from same antigens. Therefore, induction of CTLs utilizing antigen peptides is troublesome. In order to solve this problem, various attempts have been made. However, satisfactory results have not been obtained yet. One of the attempts is a T cell induction method which comprises transducing an antigen gene into a patient-derived (autogenous) antigen-presenting cell. As the antigen presenting cells, a B cell, a macrophage and a dendritic cell have been investigated. Regarding mainly a dendritic cell known as a professional antigen presenting cell, clinical tests for use as a vaccine adjuvant, etc. have been conducted. However, these antigen presenting cells have a problem that labor is required for preparing a necessary amount for immune induction. Although B cells can be prepared in a large amount by immortalization using EB viruses, there is a safety problem due to use of viruses.

Examples of a tumor antigen-specific TCR gene include genes of HLA-A02-restricted MART1-specific TCR [Nonpatent Literature 1], MAGE-A3-specific TCR [Nonpatent Literature 2], gp100-specific TCR [Nonpatent Literature 3], NY-ESO-1-specific TCR [Nonpatent Literature 4], HLA-A24-restricted WT1 (Wilms tumor 1)-specific TCR [Nonpatent Literature 5], and MAGE-A4-specific TCR [Patent Literature 1], which have been cloned.

It can be expected that cytotoxic activity specific for a target antigen can be imparted by introducing a TCR gene into arbitrary CTL. Based on this finding, gene therapy with a TCR gene targeting a MART1 [Nonpatent Literature 6], gp100 [Nonpatent Literature 3] or mHAG HA-2 antigen [Non Patent Literature 7] has been tried.

Aurora-A kinase (Aur-A) is a protein belonging to the serine/threonine kinase family which is expressed mainly at the G2/M phase of cell cycle and controls mitotic division of cells, and is overexpressed in a variety of cancers and is involved in poor prognosis. The overexpression of Aur-A is widely observed in hematopoietic tumors. The overexpression of Aur-A is associated with abnormality of centrosome duplication, abnormality of chromosome number, and instability of chromosomes. In addition, the ectopic overexpression of Aur-A leads to immortalization of rodent fibroblasts at a high rate. Accordingly, Aur-A has drawn attention as a promising target antigen for cancer vaccine therapy, and an HLA-A*0201-restricted and Aur-A₂₀₇₋₂₁₅ peptide-specific CTL clone has been obtained [Non Patent Literature 8].

### CITATION LIST

### Patent Literature

Patent Literature 1: WO 2007/032255

### Nonpatent Literature

Nonpatent Literature 1: Cancer Res., vol. 54, pp. 5265-5268 (1994)
Nonpatent Literature 2: Anticancer Res., vol. 20, pp. 1793-1799 (2000)
Nonpatent Literature 3: J. Immunol., vol. 170, pp. 2186-2194 (2003)
Nonpatent Literature 4: J. Immunol., vol. 174, pp. 4415-4423 (2005)
Nonpatent Literature 5: Blood, vol. 106, pp. 470-476 (2005)
Nonpatent Literature 6: J. Immunol., vol. 163, pp. 507-513 (1999)
Nonpatent Literature 7: Blood, vol. 103, pp. 3530-3540 (2003)
Nonpatent Literature 8: Blood, vol. 113, pp. 66-74 (2009)

### SUMMARY OF THE INVENTION

### Problems to be Solved by the Invention

As an HLA-A02-restricted TCR gene for a tumor-associated antigen, genes for MART1 or MAGE-A4 are known. From the viewpoint of effective treatment of a variety of cancers, discovery of new TCR genes for various cancer antigens which are specifically expressed in tumors is desired.

A cell having responsibility for cytotoxic activity via a TCR in a living body is a CTL having an antigen-specific TCR. In order to proliferate the CTL having an antigen-specific TCR ex vivo and use it in treating a disease such as cancer, problems in handling of the cell, for example, collection, expansion culture, etc. need to be overcome. Therefore, there is a need to provide a TCR gene specific for a tumor antigen, etc. for easily preparing a large amount of a CTL having the desired antigen specificity.

### Solution to Problems

The present inventors intensively studied CTLs against tumor antigens, and as a result, succeeded in cloning of cDNA encoding TCR α-chain and β-chain from an HLA-A*0201-restricted CTL against a tumor antigen, Aur-A. Further, the present inventors added an Aur-A₂₀₇₋₂₁₅ peptide to cells expressing an HLA-A*0201 molecule, and as a result, found that these cells exhibit HLA-A*0201-restricted and Aur-A₂₀₇₋₂₁₅ peptide-specific cytotoxicity. Thus, the present invention was completed.

A first aspect of the present invention relates to a nucleic acid encoding a polypeptide which composes an HLA-A*0201-restricted and Aur-A₂₀₇₋₂₁₅-specific T cell receptor, wherein the nucleic acid has a base sequence encoding a variable region polypeptide of the receptor. In the first aspect, an example of the nucleic acid of the present invention is a nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 2 of Sequence Listing, wherein the nucleic acid is selected from the group consisting of:

(1) a nucleic acid comprising a base sequence encoding a polypeptide consisting of the amino acid sequence set forth in SEQ ID NO: 5 of Sequence Listing,

(2) a nucleic acid comprising a base sequence encoding a polypeptide consisting of the amino acid sequence set forth in SEQ ID NO: 5 of Sequence Listing in which one to several amino acid residues are deleted, added, inserted or substituted,

(3) a nucleic acid comprising the base sequence set forth in SEQ ID NO: 6 of Sequence Listing, and

(4) a nucleic acid which can hybridize with a nucleic acid consisting of the base sequence set forth in SEQ ID NO: 6 of Sequence Listing or a complementary strand thereof under stringent conditions. The first aspect of the present invention also includes the nucleic acid which encodes a polypeptide consisting of the amino acid sequence set forth in SEQ ID NO: 1 of Sequence Listing, or a polypeptide consisting of the amino acid sequence set forth in SEQ ID NO: 1 of Sequence Listing in which one to several amino acid residues are deleted, added, inserted or substituted.

In the first aspect, another example of the nucleic acid of the present invention is a nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 1 of Sequence Listing, wherein the nucleic acid is selected from the group consisting of:

(1) a nucleic acid comprising a base sequence encoding a polypeptide consisting of the amino acid sequence set forth in SEQ ID NO: 7 of Sequence Listing,

(2) a nucleic acid comprising a base sequence encoding a polypeptide consisting of the amino acid sequence set forth in SEQ ID NO: 7 of Sequence Listing in which one to several amino acid residues are deleted, added, inserted or substituted,

(3) a nucleic acid comprising the base sequence set forth in SEQ ID NO: 8 of Sequence Listing, and

(4) a nucleic acid which can hybridize with a nucleic acid consisting of the base sequence set forth in SEQ ID NO: 8 of Sequence Listing or a complementary strand thereof under stringent conditions. The first aspect of the present invention also includes the nucleic acid which encodes a polypeptide consisting of the amino acid sequence set forth in SEQ ID NO: 2 of Sequence Listing, or a polypeptide consisting of the amino acid sequence set forth in SEQ ID NO: 2 of Sequence Listing in which one to several amino acid residues are deleted, added, inserted or substituted.

A second aspect of the present invention relates to a polypeptide encoded by the nucleic acid of the first aspect of the present invention. The polypeptide composes an HLA-A*0201-restricted and Aur-A₂₀₇₋₂₁₅-specific T cell receptor, and has a variable region polypeptide of the α-chain or β-chain of the receptor. The present invention also includes an HLA-A*0201-restricted and Aur-A₂₀₇₋₂₁₅-specific T cell receptor which is composed of the polypeptides of the second aspect of the present invention.

A third aspect of the present invention relates to a recombinant nucleic acid comprising the nucleic acid of the present invention.

A fourth aspect of the present invention relates to a vector in which at least one of the recombinant nucleic acids of the third aspect of the present invention is inserted.

The fourth aspect of the present invention also relates to a vector in which are inserted both a recombinant nucleic acid containing a nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 2 of Sequence Listing, wherein the nucleic acid is selected from the group consisting of:

(1) a nucleic acid comprising a base sequence encoding a polypeptide consisting of the amino acid sequence set forth in SEQ ID NO: 5 of Sequence Listing,

(2) a nucleic acid comprising a base sequence encoding a polypeptide consisting of the amino acid sequence set forth in SEQ ID NO: 5 of Sequence Listing in which one to several amino acid residues are deleted, added, inserted or substituted,

(3) a nucleic acid comprising the base sequence set forth in SEQ ID NO: 6 of Sequence Listing, and

(4) a nucleic acid which can hybridize with a nucleic acid consisting of the base sequence set forth in SEQ ID NO: 6 of Sequence Listing or a complementary strand thereof under stringent conditions, or a recombinant nucleic acid containing the nucleic acid which encodes a polypeptide consisting of the amino acid sequence set forth in SEQ ID NO: 1 of Sequence Listing, or a polypeptide consisting of the amino acid sequence set forth in SEQ ID NO: 1 of Sequence Listing in which one to several amino acid residues are deleted, added, inserted or substituted; and a recombinant nucleic acid containing a nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 1 of Sequence Listing, wherein the nucleic acid is selected from the group consisting of:

(1) a nucleic acid comprising a base sequence encoding a polypeptide consisting of the amino acid sequence set forth in SEQ ID NO: 7 of Sequence Listing,

(2) a nucleic acid comprising a base sequence encoding a polypeptide consisting of the amino acid sequence set forth in SEQ ID NO: 7 of Sequence Listing in which one to several amino acid residues are deleted, added, inserted or substituted,

(3) a nucleic acid comprising the base sequence set forth in SEQ ID NO: 8 of Sequence Listing, and

(4) a nucleic acid which can hybridize with a nucleic acid consisting of the base sequence set forth in SEQ ID NO: 8 of Sequence Listing or a complementary strand thereof under stringent conditions, or a recombinant nucleic acid containing the nucleic acid which encodes a polypeptide consisting of the amino acid sequence set forth in SEQ ID NO: 2 of Sequence Listing, or a polypeptide consisting of the amino acid sequence set forth in SEQ ID NO: 2 of Sequence Listing in which one to several amino acid residues are deleted, added, inserted or substituted.

The fourth aspect of the present invention also relates to a combination of a vector in which is inserted a recombinant nucleic acid containing a nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 2 of Sequence Listing, wherein the nucleic acid is selected from the group consisting of:

(1) a nucleic acid comprising a base sequence encoding a polypeptide consisting of the amino acid sequence set forth in SEQ ID NO: 5 of Sequence Listing,

(2) a nucleic acid comprising a base sequence encoding a polypeptide consisting of the amino acid sequence set forth in SEQ ID NO: 5 of Sequence Listing in which one to several amino acid residues are deleted, added, inserted or substituted,

(3) a nucleic acid comprising the base sequence set forth in SEQ ID NO: 6 of Sequence Listing, and

(4) a nucleic acid which can hybridize with a nucleic acid consisting of the base sequence set forth in SEQ ID NO: 6 of Sequence Listing or a complementary strand thereof under stringent conditions, or a recombinant nucleic acid containing the nucleic acid which encodes a polypeptide consisting of the amino acid sequence set forth in SEQ ID NO: 1 of Sequence Listing, or a polypeptide consisting of the amino acid sequence set forth in SEQ ID NO: 1 of Sequence Listing in which one to several amino acid residues are deleted, added, inserted or substituted; and a vector in which is inserted a recombinant nucleic acid containing a nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 1 of Sequence Listing, wherein the nucleic acid is selected from the group consisting of:

(1) a nucleic acid comprising a base sequence encoding a polypeptide consisting of the amino acid sequence set forth in SEQ ID NO: 7 of Sequence Listing,

(2) a nucleic acid comprising a base sequence encoding a polypeptide consisting of the amino acid sequence set forth in SEQ ID NO: 7 of Sequence Listing in which one to several amino acid residues are deleted, added, inserted or substituted,

(3) a nucleic acid comprising the base sequence set forth in SEQ ID NO: 8 of Sequence Listing, and

(4) a nucleic acid which can hybridize with a nucleic acid consisting of the base sequence set forth in SEQ ID NO: 8 of Sequence Listing or a complementary strand thereof under stringent conditions, or a recombinant nucleic acid containing a nucleic acid which encodes a polypeptide consisting of the amino acid sequence set forth in SEQ ID NO: 2 of Sequence Listing, or a polypeptide consisting of the amino acid sequence set forth in SEQ ID NO: 2 of Sequence Listing in which one to several amino acid residues are deleted, added, inserted or substituted.

A fifth aspect of the present invention relates to a cell expressing an HLA-A*0201-restricted and Aur-A₂₀₇₋₂₁₅-specific TCR in which the vector or the combination of vectors of the fourth aspect of the present invention introduced. In the fifth aspect, an example of the cell of the present invention is a T cell or a cell which can differentiate into a T cell.

A sixth aspect of the present invention relates to a carcinostatic agent comprising the vector or the combination of vectors of the fourth aspect of the present invention or the cell of the fifth aspect of the present invention, as an active ingredient.

A seventh aspect of the present invention relates to a method of treating a cancer which comprises a step of administering the carcinostatic agent of the sixth aspect of the present invention.

An eighth aspect of the present invention relates to the vector or the combination of vectors of the fourth aspect of the present invention or the cell of the fifth aspect of the present invention for use in treatment of a cancer.

### Effects of the Invention

According to the present invention, there are provided nucleic acids each of which encodes either the α-chain or β-chain of an HLA-A*0201-restricted and Aur-A₂₀₇₋₂₁₅-specific TCR. Also, there is provided a method of damaging a tumor cell which comprises using an HLA-A*0201-unrestricted or Aur-A₂₀₇₋₂₁₅-unspecific T cell as an effector cell. The effector cell is useful, for example, in treatment of a cancer.

### BRIEF DESCRIPTION OF THE DRAWINGS

FIG. 1 shows the cytotoxic activity of a T2 cell, a TCR α-chain gene and β-chain gene-introduced CD8-positive cell and a CD8-positive cell pulsed with Aur-A₂₀₇₋₂₁₅.

FIG. 2 shows results of flow cytometry analysis of the TCR gene-introduced human CD8-positive lymphocytes of the present invention, which were allowed to react with an anti-human CD8 antibody and an anti-human Vb12 antibody.

FIG. 3 shows results of tetramer assay of the TCR gene-introduced human CD8-positive lymphocytes of the present invention.

FIG. 4 shows the Aurora-A-specific cytotoxic activity of the TCR gene-introduced human CD8-positive lymphocytes of the present invention.

FIG. 5 shows the cell number-dependent cytotoxic activity of the TCR gene-introduced human CD8-positive lymphocytes of the present invention on HLA-A*0201-positive leukemia cell strain GANMO-1.

FIG. 6 shows analysis results of cytotoxic activity of the TCR gene-introduced human CD8-positive lymphocytes of the present invention on PBMC.

FIG. 7 shows that the TCR gene-introduced human CD8-positive lymphocytes of the present invention precisely recognizes a complex in which a target Aurora-A peptide derived from a cancer antigen Aurora A protein in a leukemia cell is presented on HLA-A*0201.

### MODE FOR CARRYING OUT THE INVENTION

The first aspect of the present invention relates to nucleic acids encoding polypeptides which compose an HLA-A*0201-restricted and Aur-A₂₀₇₋₂₁₅-specific T cell receptor, wherein the nucleic acid has a base sequence encoding a variable region polypeptide of the receptor. The polypeptides comprise the two kinds of TCR α-chain and TCR β-chain. An HLA-A*0201-restricted and Aur-A₂₀₇₋₂₁₅-specific TCR is composed of both the chains in combination.

Herein, “HLA-A*0201-restricted and Aur-A₂₀₇₋₂₁₅-specific TCR” means that the TCR specifically recognizes a complex of a peptide having the amino acid sequence set forth in SEQ ID NO: 9 of Sequence Listing (Aur-A₂₀₇₋₂₁₅, hereinafter, abbreviated as P207) and an HLA-A*0201 molecule and, when the TCR is present on the T cell surface, HLA-A*0201-restricted and P207-specific cytotoxic activity on a target cell can be imparted to the T cell. The specific recognition of the complex by a T cell may be confirmed by a known method, and examples of preferred methods include tetramer analysis using an HLA-A*0201 molecule and P207, and an interferon gamma production assay. By performing the interferon gamma production assay, it can be confirmed that a T cell expressing the TCR on the cell surface recognizes a target cell via the TCR, and a signal thereof is transmitted into a cell. It may be also confirmed that cytotoxic activity can be imparted to a T cell when the complex is present on the T cell surface by using a known method, and examples of preferred methods include measurement of cytotoxic activity on an HLA-A*0201-positive target cell, such as a chromium release assay.

The α-chain polypeptide means a polypeptide which can form an HLA-A*0201-restricted and Aur-A₂₀₇₋₂₁₅-specific TCR together with the β-chain, wherein the α-chain variable region has a polypeptide selected from a polypeptide of the amino acid sequence set forth in SEQ ID NO: 5 of Sequence Listing; a polypeptide consisting of the amino acid sequence set forth in SEQ ID NO: 5 of Sequence Listing in which one to several amino acid residues are deleted, added, inserted or substituted; and a polypeptide having at least 90%, preferably at least 95% or more, more preferably 98% or more, further preferably 99% or more amino acid sequence identity with the amino acid sequence set forth in SEQ ID NO: 5 of Sequence Listing. In the present invention, a nucleic acid encoding a polypeptide derived from a TCR α-chain contains, as an essential constituent component, a base sequence encoding the amino acid sequence of the α-chain variable region or a similar sequence thereof. A preferred example of the nucleic acid of the present invention is a nucleic acid encoding a polypeptide selected from a polypeptide consisting of an amino acid sequence of the entire α-chain or a similar sequence thereof which contains the constant region, that is, the amino acid sequence set forth in SEQ ID NO: 1 of Sequence Listing; a polypeptide consisting of the amino acid sequence set forth in SEQ ID NO: 1 of Sequence Listing in which one to several amino acid residues are deleted, added, inserted or substituted; and a polypeptides having at least 90%, preferably at least 93% or more, more preferably 98% or more, further preferably 99% or more amino acid sequence identity with the amino acid sequence set forth in SEQ ID NO: 1 of Sequence Listing.

As used herein, “one to several” means 1 to 30, preferably 1 to 20, further preferably 1 to 9.

The β-chain polypeptide means a polypeptide which can form an HLA-A*0201-restricted and Aur-A₂₀₇₋₂₁₅-specific TCR together with the α-chain, wherein the β-chain variable region has a polypeptide selected from a polypeptide of the amino acid sequence set forth in SEQ ID NO: 7 of Sequence Listing; a polypeptide consisting of the amino acid sequence set forth in SEQ ID NO: 7 of Sequence Listing in which one to several amino acid residues are deleted, added, inserted or substituted; and a polypeptide having at least 90%, preferably at least 95% or more, more preferably 98% or more, further preferably 99% or more amino acid sequence identity with the amino acid sequence set forth in SEQ ID NO: 7 of Sequence Listing. In the present invention, a nucleic acid encoding a polypeptide derived from a TCR β-chain contains, as an essential constituent component, a base sequence encoding the amino acid sequence of the β-chain variable region or a similar sequence thereof. A preferred example of the nucleic acid of the present invention is a nucleic acid encoding a polypeptide selected from a polypeptide consisting of an amino acid sequence of the entire β-chain or a similar sequence thereof which contains the constant region, that is, the amino acid sequence set forth in SEQ ID NO: 2 of Sequence Listing; a polypeptide consisting of the amino acid sequence set forth in SEQ ID NO: 2 of Sequence Listing in which one to several amino acid residues are deleted, added, inserted or substituted; and a polypeptides having at least 90%, preferably at least 93% or more, more preferably 98% or more, further preferably 99% or more amino acid sequence identity with the amino acid sequence set forth in SEQ ID NO: 2 of Sequence Listing.

Examples of the nucleic acid encoding the α-chain polypeptide include, but not limited to, a nucleic acid consisting of the base sequence set forth in SEQ ID NO: 3 of Sequence Listing, and a nucleic acid which can hybridize with a nucleic acid of the aforementioned base sequence or a complementary strand thereof under stringent conditions. Examples of the nucleic acid encoding the variable region of the α-chain polypeptide include a nucleic acid consisting of the base sequence set forth in SEQ ID NO: 6 of Sequence Listing, and a nucleic acid which hybridizes with a nucleic acid of the aforementioned base sequence or a complementary strand thereof under stringent conditions and encodes a polypeptide capable of forming an HLA-A*0201-restricted and Aur-A₂₀₇₋₂₁₅-specific TCR together with a β-chain. Examples of the nucleic acid encoding the β-chain polypeptide include a nucleic acid consisting of the base sequence set forth in SEQ ID NO: 4 of Sequence Listing, and a nucleic acid which hybridizes with a nucleic acid of the aforementioned base sequence or a complementary strand thereof under stringent conditions and can form an HLA-A*0201-restricted and Aur-A₂₀₇₋₂₁₅-specific TCR together with an α-chain. Examples of the nucleic acid encoding the variable region of the β-chain polypeptide include a nucleic acid consisting of the base sequence set forth in SEQ ID NO: 8 of Sequence Listing, and a nucleic acid which can hybridize with a nucleic acid of the aforementioned base sequence or a complementary strand thereof under stringent conditions.

Herein, examples of the stringent conditions include conditions described in Molecular Cloning: A Laboratory Manual 2^{nd} ed., published by Cold Spring Harbor Laboratory in 1989, edited by J. Sambrook et al., etc. Specifically, an example of the stringent conditions comprises incubation of a nucleic acid with a probe at 65° C. for 12 to 20 hours in 6×SSC containing 0.5% SDS, 5×Denhardt's solution, and 0.01% modified salmon sperm DNA. The nucleic acid hybridized with the probe can be washed in 0.1×SSC containing 0.5% SDS at 37° C. to remove the nonspecifically bound probe and then detected.

A nucleic acid as used herein means a single-stranded or double-stranded DNA, RNA or DNA-RNA chimera, or a DNA-RNA hetero duplex. When all or a part of the nucleic acid is RNA, regarding the partial sequence of RNA, T is read as U in Sequence Listing described herein. Examples of a preferable aspect of the present invention include a combination of two kinds of nucleic acids, consisting of the nucleic acid encoding the TCR α-chain polypeptide of the present invention or the nucleic acid encoding a polypeptide having the TCR α-chain variable region polypeptide of the present invention, and the nucleic acid encoding the TCR β-chain polypeptide of the present invention or the nucleic acid encoding a polypeptide having the TCR β-chain variable region polypeptide of the present invention. The combination of the nucleic acids is useful for the purpose of expressing an HLA-A*0201-restricted and Aur-A₂₀₇₋₂₁₅-specific TCR in a cell.

The nucleic acid of the present invention can be obtained, for example, as described below. An RNA is prepared from an HLA-A*0201-restricted and Aur-A₂₀₇₋₂₁₅-specific CTL, for example, a clone #AUR-1 described in Nonpatent Literature 8 by a conventional method, and then a cDNA is synthesized. Using the cDNA as a template, 5′-rapid amplification of cDNA end (RACE) is performed using an antisense primer complementary to a nucleic acid encoding the TCR α-chain constant region and an antisense primer complementary to the β-chain constant region. 5′-RACE may be performed by a known method. For example, 5′-RACE can be performed using a commercially available kit such as SMART PCR cDNA Synthesis Kit (manufactured by Clontech). A DNA amplified by the aforementioned procedure is incorporated into a plasmid vector, and Escherichia coli is transformed with the vector. A plasmid is prepared from the transformant, and the base sequence of the inserted DNA is determined.

By comparing the resulting base sequence with the known sequence of a TCR α-chain or β-chain gene, a DNA amplified by 5′-RACE and having no relationship with a TCR gene can be excluded. In addition, since there is a possibility that a DNA having the base sequence into which mutation was introduced during PCR is amplified by PCR, it is preferable that sequencing is performed from a plurality of Escherichia coli clones, the consensus sequence thereof is determined, and the sequences of a TCR α-chain gene and a TCR β-chain gene originally possessed by the CTL are presumed from the consensus sequence, and then used in the present invention.

A nucleic acid having the base sequence set forth in SEQ ID NO: 3 of Sequence Listing which encodes the TCR α-chain of the amino acid sequence of SEQ ID NO: 1, and a nucleic acid having the base sequence set forth in SEQ ID NO: 4 of Sequence Listing which encodes the TCR β-chain of the amino acid sequence of SEQ ID NO: 2 are obtained by the aforementioned method.

In the present invention, a DNA obtained by the aforementioned method may be used, or a nucleic acid having the same sequence as that of the DNA may be chemically synthesized and used. Further, a DNA which has a base sequence different from that of the DNA and encodes the same amino acid sequence as the DNA encodes can be also made and used. Upon design of a DNA having a base sequence different from that of the DNA, it is possible to select codons to be used, based on codon usage in a cell and other purposes.

A known method of obtaining a T cell expressing a TCR capable of recognizing a target antigen at a high ratio comprises suppressing the expression of the endogenous TCR α-chain and TCR β-chain which a T cell originally expresses by siRNA when nucleic acids encoding TCR α-chain and β-chain capable of recognizing a target antigen are introduced into the T cell (see WO 2008/153029). When the nucleic acid of the present invention is applied to the aforementioned method, the T cell o expressing a TCR at a high ratio of the present invention can be obtained by rendering the base sequence of the nucleic acid of the present invention a sequence different from the base sequence corresponding to an RNA on which siRNA acts to suppress the expression of the endogenous TCR α-chain and TCR β-chain. The base sequence can be made by introducing silent mutation into the naturally-occurring nucleic acid of the present invention, or by chemically synthesizing an artificially designed nucleic acid.

Of the nucleic acids of the present invention, a nucleic acid encoding the part corresponding to the variable region of each of both chains composing a TCR can be connected to a region encoding a constant region, an intracellular region, etc., within a nucleic acid encoding another functional molecule, for example, an antibody, a receptor, etc. The novel nucleic acid thus constructed is useful in producing a chimera functional molecule to which HLA-A*0201-restricted and Aur-A₂₀₇₋₂₁₅-specific binding activity has been imparted.

Using the nucleic acid of the present invention, polypeptides composing an HLA-A*0201-restricted and Aur-A₂₀₇₋₂₁₅-specific TCR can be produced in a genetic engineering manner. For example, a cell can be made to express an HLA-A*0201-restricted and Aur-A₂₀₇₋₂₁₅-specific TCR by introducing both a nucleic acid encoding the α-chain polypeptide and a nucleic acid encoding the β-chain into the cell to express both the peptides.

In other words, the second aspect of the present invention relates to polypeptides composing an HLA-A*0201-restricted and Aur-A₂₀₇₋₂₁₅-specific T cell receptor, wherein the polypeptide is encoded by the nucleic acid of the first aspect of the present invention and has a polypeptide of a variable region of the α-chain or β-chain of the receptor.

The α-chain polypeptide means a polypeptide which can form an HLA-A*0201-restricted and Aur-A₂₀₇₋₂₁₅-specific TCR together with the β-chain, wherein the α-chain variable region has a polypeptide selected from a polypeptide of the amino acid sequence set forth in SEQ ID NO: 5 of Sequence Listing; a polypeptide consisting of the amino acid sequence set forth in SEQ ID NO: 5 of Sequence Listing in which one to several amino acid residues are deleted, added, inserted or substituted; and a polypeptide having at least 90%, preferably at least 95% or more, more preferably 98% or more, further preferably 99% or more amino acid sequence identity with the amino acid sequence set forth in SEQ ID NO: 5 of Sequence Listing. A preferred example of the polypeptide of the present invention is a polypeptide selected from a polypeptide consisting of an amino acid sequence of the entire α-chain or a similar sequence thereof which contains the constant region, that is, the amino acid sequence set forth in SEQ ID NO: 1 of Sequence Listing; a polypeptide consisting of the amino acid sequence set forth in SEQ ID NO: 1 of Sequence Listing in which one to several amino acid residues are deleted, added, inserted or substituted; and a polypeptides having at least 90%, preferably at least 93% or more, more preferably 98% or more, further preferably 99% or more amino acid sequence identity with the amino acid sequence set forth in SEQ ID NO: 1 of Sequence Listing.

The β-chain polypeptide means a polypeptide which can form an HLA-A*0201-restricted and Aur-A₂₀₇₋₂₁₅-specific TCR together with the α-chain, wherein the β-chain variable region has a polypeptide selected from a polypeptide of the amino acid sequence set forth in SEQ ID NO: 7 of Sequence Listing; a polypeptide consisting of the amino acid sequence set forth in SEQ ID NO: 7 of Sequence Listing in which one to several amino acid residues are deleted, added, inserted or substituted; and a polypeptide having at least 90%, preferably at least 95% or more, more preferably 98% or more, further preferably 99% or more amino acid sequence identity with the amino acid sequence set forth in SEQ ID NO: 7 of Sequence Listing. A preferred example of the polypeptide of the present invention is a polypeptide selected from a polypeptide consisting of an amino acid sequence of the entire β-chain or a similar sequence thereof which contains the constant region, that is, the amino acid sequence set forth in SEQ ID NO: 2 of Sequence Listing; a polypeptide consisting of the amino acid sequence set forth in SEQ ID NO: 2 of Sequence Listing in which one to several amino acid residues are deleted, added, inserted or substituted; and a polypeptides having at least 90%, preferably at least 93% or more, more preferably 98% or more, further preferably 99% or more amino acid sequence identity with the amino acid sequence set forth in SEQ ID NO: 2 of Sequence Listing.

Further, an HLA-A*0201-restricted and Aur-A₂₀₇₋₂₁₅-specific TCR which is composed of the polypeptides of the present invention is also included in the present invention. Although it is not particularly intended to limit the present invention, the TCR can be prepared in an isolated form from biological components which the TCR is associated with in nature, for example, by using the nucleic acid of the present invention to artificially express a polypeptide encoded by the nucleic acid.

The third aspect of the present invention is a recombinant nucleic acid comprising the nucleic acid of the present invention. Examples of the recombinant nucleic acid include, but not limited to, a nucleic acid to which a variety of elements enabling the translation of a polypeptide encoded by the nucleic acid of the present invention when the nucleic acid is introduced into a cell are added.

Examples of the recombinant nucleic acid of the present invention comprising a DNA include recombinant nucleic acids having a promoter (e.g., mammal-derived promoters such as phosphoglycerate kinase promoter, Xist promoter, β-actin promoter, RNA polymerase II promoter, etc., virus-derived promoters such as SV40 early promoter, cytomegalovirus promoter, thymidine kinase promoter of herpes simplex virus, LTR promoters of various retroviruses, etc.), a terminator, an enhancer, or other transcription control regions. Further, the recombinant nucleic acid may have a sequence which contributes to the translation of a polypeptide encoded by the nucleic acid of the first invention (Kozak sequence, etc.). Of course, the aforementioned elements are placed at functionally associated positions with each other so as to be suitable for the transcription of the nucleic acid of the present invention to an RNA or the translation of a polypeptide. In the case where the recombinant nucleic acid is an RNA, elements relating to transcription control may not be added to the recombinant nucleic acid of the present invention.

The recombinant nucleic acid of the present invention can be incorporated into a vector as described later. In addition, the recombinant nucleic acid of the present invention which is an RNA can be also introduced directly into a cell to express a TCR. As a method of introduction of an RNA, a known method may be used and, for example, an electroporation method can be suitably used.

The fourth aspect of the present invention relates to a vector in which at least one of the recombinant nucleic acids of the present invention is inserted. The vector is useful in making a desired cell express an HLA-A*0201-restricted and Aur-A₂₀₇₋₂₁₅-specific TCR. Particularly preferred examples of the vector of the present invention include (1) a vector in which both the recombinant nucleic acid of the present invention which contains a nucleic acid encoding the TCR α-chain polypeptide or a polypeptide having the variable region polypeptide of the TCR α-chain, and the recombinant nucleic acid of the present invention which contains a nucleic acid encoding the TCR β-chain polypeptide or a polypeptide having the variable region polypeptide of the TCR β-chain polypeptide are inserted, and (2) a combination of a vector in which the recombinant nucleic acid of the present invention which contains a nucleic acid encoding the TCR α-chain polypeptide or a polypeptide having the variable region polypeptide of the TCR α-chain polypeptide is inserted, and a vector in which the recombinant nucleic acid of the present invention which contains a nucleic acid encoding the TCR β-chain polypeptide or a polypeptide having the variable region polypeptide of the TCR β-chain polypeptide is inserted. In the aspect of (1), the nucleic acid encoding the TCR α-chain polypeptide and the nucleic acid encoding the TCR β-chain polypeptide may be transcribed and translated by separate promoters, or may be transcribed and translated by one promoter using an internal ribosome entry site (IRES) or a self-digesting 2A peptide.

The vector of the present invention may further have a sequence contributing to the expression of siRNA for suppressing the expression of the endogenous TCR α-chain and/or TCR β-chain which a T cell originally expresses.

A vector used in the present invention is not particularly limited, and a suitable vector may be selected and then used from known vectors such as a plasmid vector and a virus vector depending on the purpose. For example, when the recombinant nucleic acid is incorporated into a plasmid vector, a gene introduction method such as a calcium phosphate method, a cationic lipid method, a liposome method, an electroporation method, etc., can be used for introduction of the vector into a cell.

A virus vector having the ability to infect a cell to introduce a foreign DNA into the cell is suitable in the present invention. In the present invention, known virus vectors such as a retrovirus vector (including lentivirus vector, pseudo-type vector, etc.), an adenovirus vector, an adeno-associated virus vector, a herpesvirus vector, etc., can be used. A virus vector in which the recombinant nucleic acid of the present invention is inserted makes it possible to infect a target cell under the conditions suitable for each virus, and to introduce the nucleic acid of the present invention into the cell. A retrovirus vector having the ability to incorporate an inserted foreign nucleic acid onto a chromosome is suitable in the present invention.

The fifth aspect of the present invention relates to a cell expressing an HLA-A*0201-restricted and Aur-A₂₀₇₋₂₁₅-specific TCR, wherein the nucleic acid of the present invention is introduced in the cell. Herein, the nucleic acid of the present invention may be introduced into a desired cell as the recombinant nucleic acid of the present invention or the vector of the present invention. Preferred examples of the cell of the present invention include a cell in which both a nucleic acid encoding the TCR α-chain polypeptide or a polypeptide having the variable region polypeptide of the TCR α-chain, and a nucleic acid encoding the TCR β-chain polypeptide or a polypeptide having the variable region polypeptide of the TCR β-chain polypeptide are introduced, and a cell transformed with the vector of the present invention. The present invention also includes a cell in which the aforementioned nucleic acids are incorporated onto a chromosomal DNA.

Since a TCR plays an important role in recognition of an antigen by a T cell, a preferred aspect of the present invention is a T cell in which the nucleic acid of the present invention is introduced. A T cell expressing an HLA-A*0201-restricted and Aur-A₂₀₇₋₂₁₅-specific TCR can be obtained by taking a T cell from a living body and then introducing the nucleic acid of the present invention into the T cell by the aforementioned procedure. Further, in a preferred aspect of the present invention, the nucleic acid may be introduced into a cell capable of differentiating into a T cell and, thereafter, the cell may be differentiated into a T cell. Examples of the cell capable of differentiating into a T cell include a hematopoietic stem cell, a common lymphoid progenitor, and a T cell progenitor. In addition, it is not necessary that a subject cell into which a nucleic acid is introduced is fractionated into a single cell species. A cell population containing the subject cell, for example, a peripheral blood mononuclear cell can be used as a subject into which a nucleic acid is introduced.

The cell population containing the subject cell into which a nucleic acid is introduced may be collected from, for example, peripheral blood, bone marrow or umbilical blood of a human or a non-human mammal. If necessary, a T cell and/or a cell capable of differentiating into a T cell can be fractionated or enriched and then used in the present invention. When the TCR gene-introduced cell of the present invention is used in treating a cancer, etc., it is preferable that the cell population is collected from a patient to be treated, or a donor having an HLA type matched with that of the patient.

A method of introducing the nucleic acid of the present invention into a cell is not particularly limited, and can be a known method. When the nucleic acid of the present invention or the recombinant nucleic acid of the present invention is introduced, for example, a method using an electroporation method, a calcium phosphate method, a cationic lipid method or a liposome method can be used. The nucleic acid can be easily introduced into a cell with high efficiency by using a commercially available transfection reagent [e.g., TransIT series (manufactured by Mirus), GeneJuice (manufactured by Novagen), RiboJuice (manufactured by Novagen), Lipofectamine (manufactured by Invitrogen)]. When the vector of the present invention is used, if the vector is a plasmid vector, it can be introduced into a cell by the same method as that for a nucleic acid. If the vector is a virus vector, a suitable infection method for each virus vector may be selected. Particularly, when a retrovirus vector is used, use of CH-296 (manufactured by TAKARA BIO INC.) which is a recombinant fibronectin fragment allows for highly efficient gene introduction into various cells, particularly, into a hematopoietic stem cell having a low infection efficiency of a retrovirus vector.

The sixth aspect of the present invention relates to a carcinostatic agent comprising, as an active ingredient, the vector of the fourth aspect of the present invention or the cell of the fifth aspect of the present invention. AT cell in which the nucleic acid of the present invention introduced, obtained according to the fifth aspect of the present invention exhibits cytotoxic activity on a cell presenting an HLA-A*0201 molecule and an Aur-A₂₀₇₋₂₁₅ peptide. Therefore, the vector and cell of the present invention can be used as a carcinostatic agent for a cancer expressing Aur-A.

The carcinostatic agent of the present invention contains the vector or cell of the present invention as an active ingredient. The carcinostatic agent is provided, for example, in the form of suspension of the vector or the cell in a pharmaceutically acceptable diluent. As used herein, examples of the diluent include a medium suitable for keeping the vector or the cell, a physiological saline, and a phosphate-buffered physiological saline. Examples of the medium generally include, but not limited to, media such as RPMI, AIM-V, and X-VIVO10. The carcinostatic agent may further contain a pharmaceutical acceptable carrier, preservative, etc. for the purpose of stabilization. As used herein, examples of the carrier include human serum albumin, etc. The carcinostatic agent containing the cell of the present invention as an active ingredient may contain the cells in an amount of preferably 1×10⁴ to 1×10⁸ cells/mL, more preferably 5×10⁵ to 5×10⁷ cells/mL.

When the carcinostatic agent containing the cell of the present invention as an active ingredient is administered to human, it can be administered, for example, with a syringe, and a dose is preferably 1×10⁶ to 1×10¹⁰ cells per adult. The aforementioned dose is a rough indication, and a dose of the carcinostatic agent is not limited thereto. In the case of the carcinostatic agent containing the vector of the present invention as an active ingredient, the vector concentration contained in the carcinostatic agent and a dose of the carcinostatic agent may be greatly varied depending on the administration route, the kind of the vector, etc.

As described above, a method of treating a cancer is provided according to the present invention. When the vector of the fourth aspect of the present invention is used as an active ingredient, the treating method is in vivo gene therapy. On the other hand, when the cell of the present invention is used as an active ingredient, the treating method is ex vivo gene therapy, in which a nucleic acid encoding an HLA-A*0201-restricted and Aur-A₂₀₇₋₂₁₅-specific TCR is introduced into a cell separated from the body, and the cell is then administered to a patient. In the treating method of the present invention, a nucleic acid can be introduced into a cell derived from a patient to be administered (e.g., a mammal, preferably a human) or a cell derived from an individual having the same type of HLA as the patient has, and therefore, toxicity is not particularly found.

The present invention also relates to the vector or combination of vectors of the fourth aspect of the present invention or the cell of the fifth aspect of the present invention for use in treatment of a cancer.

### EXAMPLES

The present invention will be further explained more in more detail by way of Examples, which the present invention is not limited to.

### Example 1

### Cloning and Sequencing of TCR α-Chain and β-Chain Genes of Aur-A-Specific CTL Clone AUR-1

(1) Preparation of RNA from AUR-1,5′-RACR, Cloning

An AUR-1 cell which is a CTL clone exhibiting HLA-A*0201-restricted cytotoxic activity on a target cell pulsed with an Aur-A₂₀₇₋₂₁₅ peptide (SEQ ID NO: 9 of Sequence Listing, hereinafter, abbreviated as P207) described in Nonpatent Literature 8 was cultured, and RNA was extracted from 2×10⁶ cells using RNeasy Mini Kit (manufactured by Qiagen). Using 400 ng of the RNA as a template, cDNA was synthesized using CapFishing Full-length cDNA Premix Kit (manufactured by Seegene) according to the instruction of the kit. In the reverse transcription reaction, an oligo-dT adaptor set forth in SEQ ID NO: 10 of Sequence Listing, Reverse Transcriptase M-MLV (RNaseH free) (manufactured by TAKARA BIO INC.) and a reaction buffer attached to the aforementioned enzyme were used.

Using the single-stranded a cDNA thus obtained as a template, PCR was performed using the aforementioned kit. As a 5′-primer, a 5′-RACE primer (SEQ ID NO: 11) attached to the kit was used and, as a 3′-primer, a 3-TRα-C primer (SEQ ID NO: 12) which is specific for a TCR α-chain C region, a 3-TRβ-C1 primer (SEQ ID NO: 13) which is specific for a TCR β-chain C1 region, or a 3-TRβ-C2 primer (SEQ ID NO: 14) which is specific for a TCR β-chain C2 region was used. These reactions are referred to as PCR-α, PCR-β1 and PCR-β2, respectively. After each reaction solution was retained at 94° C. for 3 minutes, a cycle of 40 seconds at 94° C., 40 seconds at 58° C. and 1 minute at 72° C. was repeated 30 times, and then, the reaction solution was retained at 72° C. for 5 minutes. When a part of each reaction product was analyzed by agarose gel electrophoresis, about 1 kb of a DNA was amplified in PCR-α and PCR-β1.

DNAs in the remainders of the PCR-α and PCR-β1 reaction products were separated by agarose gel electrophoresis, and then, about 1 kb of DNA was recovered from the gel. The DNA was inserted into pMD20 (manufactured by TAKARA BIO INC.), and the resulting recombinant plasmid was used to transform Escherichia coli DH5α.

### (2) Sequencing, Clustering, and Selection of Clone

From the PCR-α or PCR-β1-derived transformants thus obtained, 96 transformants were selected. A plasmid was prepared from each transformant, and then subjected to sequencing with an automatic sequencer to determine the DNA base sequence of the plasmid. When clustering was performed after removal of the sequence of pMD20 from the sequence data, the sequences of the longest open reading frames contained in the consensus sequence of the greatest contig were as set forth in SEQ ID NO: 3 and SEQ ID NO: 4 of Sequence Listing. These sequences are the cDNA sequences of TCR α-chain gene and β-chain gene of the AUR-1 cell, respectively. The amino acid sequences of TCR α-chain and β-chain presumed from the cDNA base sequences are set forth in SEQ ID NO: 1 and SEQ ID NO: 2 of Sequence Listing. From the plasmids, plasmids having the consensus sequence of TCR α-chain gene or β-chain gene cDNA were selected and were named as pMDT-AR-TCR-A and pMDT-AR-TCR-B, respectively. Amino acid sequences of the α-chain and β-chain variable regions are set forth in SEQ ID NO: 5 and SEQ ID NO: 7 of Sequence Listing, respectively. Nucleotide sequences encoding the variable regions in the α-chain and β-chain cDNAs are set forth in SEQ ID No: 6 and SEQ ID No: 8 of Sequence Listing, respectively.

### Example 2

### Expression of Aur-A-Specific TCR by Gene Introduction Using TCR α-Chain Gene and β-Chain Gene-Expressing Retrovirus Vector

### (1) Construction of TCR α-Chain Gene and β-Chain Gene-Expressing Retrovirus Vector Plasmid

Using pMSCVneo (manufactured by Clontech) as a template, PCR was performed using a 5′ primer (SEQ ID NO: 15) with a recognition sequence of the restriction enzyme Xho I and a 3′ primer (SEQ ID NO: 16) with a recognition sequence of the restriction enzyme Eco RI, and a 3′-LTR site was amplified, and cut with Xho I (manufactured by TAKARA BIO INC.) and Eco RI (manufactured by TAKARA BIO INC.). The resulting fragments were cloned into the Xho I-Eco RI site of a pMT vector [pM vector described in Gene Therapy, vol. 7, pp. 797-804 (2000)] to prepare a pMT-MS vector. An about 340 bp fragment was obtained by cutting a pMEI-5 vector (manufactured by TAKARA BIO INC.) with the restriction enzymes Mlu I and Xho I and cloned into the Mlu I-Xho I site of the pMT-MS vector to prepare a pMS3-MC vector.

Using pMDT-AR-TCR-A as a template, PrimeSTAR (registered trademark) DNA Polymerase HS (manufactured by TAKARA BIO INC.) and primers set forth in SEQ ID NO: 17 and SEQ ID NO: 18, PCR was performed by repeating 30 times a cycle of 10 seconds at 98° C., 5 seconds at 55° C. and 1 minute at 72° C. to obtain about 1 kb of an amplified fragment. The fragment was purified from the reaction solution and digested with Not I (manufactured by TAKARA BIO INC.) and Bam HI (manufactured by TAKARA BIO INC.). The digestion fragment was named as Aurora-TCR-A insert. Separately, using pMDT-AR-TCR-B as a template, PrimeSTAR (registered trademark) DNA polymerase HS and primers set forth in SEQ ID NO: 19 and SEQ ID NO: 20, PCR was performed by repeating 30 times a cycle of 10 seconds at 98° C., 5 seconds at 55° C. and 1 minute at 72° C. to obtain an about 1 kb of an amplified fragment. The fragment was purified from the reaction solution and digested with Not I and Bam HI. The digestion fragment was named as Aurora-TCR-B insert. Plasmids obtained by inserting the Aurora-TCR-A insert and the Aurora-TCR-B insert into pMS3-MC which had been digested with Not I and Bam HI were named as pMS3-AR-A and pMS3-AR-B, respectively.

A genome was extracted from a PG13 cell, and subjected to PCR using PrimeSTAR (registered trademark) DNA Polymerase HS and primers set forth in SEQ ID NO: 21 and SEQ ID NO: 22 which comprises repeating 30 times a cycle of 10 seconds at 98° C., 5 seconds at 55° C. and 30 seconds at 72° C. A DNA in the reaction product was purified, and digested with Bgl II (manufactured by TAKARA BIO INC.) and Xho I. The digestion product was named as mPGK-Bgl II/Not I. About 1 kb of a DNA obtained by digesting the pMS3-AR-A with Not I and Xho I was separated and recovered by agarose gel electrophoresis, and named as AR-TCR-A-Not I/Xho I. Three of the prepared mPGK-Bgl II/Not I, the prepared AR-TCR-A-Not I/Xho I, and the pMS3-AR-B digested with Bam HI and Xho I were mixed, and ligated. The resulting plasmid was named as pMS3-AR-bPa.

### (2) Preparation of TCR α-Chain Gene and β-Chain Gene-Expressing Retrovirus Vector

A G3T-hi cell (manufactured by TAKARA BIO INC.) was transfected with the pMS3-AR-bPa using Retrovirus Packaging Kit Eco (manufactured by TAKARA BIO INC.) to transiently obtain ecotropic retrovirus-MS3-AR-bPa. A PG13 cell was infected with the resulting ecotropic retrovirus 3 times to prepare a bulk MS3-AR-bPa-producing cell. By using the prepared bulk MS3-AR-bPa-producing cell, a GaLV-MS3-AR-bPa retrovirus which is a GaLV envelope retrovirus was recovered. Separately, a G3T-hi cell was transfected with the pMS3-AR-bPa using Retrovirus Packaging Kit Ampho (manufactured by TAKARA BIO INC.) to transiently obtain amphotropic retrovirus-MS3-AR-bPa. The resulting retrovirus solution was filtered with a 0.45 μm filter (Milex HV, manufactured by Millipore), and stored in an ultralow temperature freezer at −80° C. until use.

(3) Introduction of Gene into Cell

To wells of a 24-well plate coated with RetroNectin (registered trademark, manufactured by TAKARA BIO INC.) was added 500 microliters of a 5-fold diluted solution of the amphotropic retrovirus-MS3-AR-bPa, and was allowed to stand at 37° C. for 4 hours. Thereafter, the virus solution was removed, and the wells were washed with PBS. To the wells were added 1×10⁵ SupT1 cells, and virus infection was performed.

Separately, to wells of a 24-well plate coated with RetroNectin (registered trademark) was added 600 microliters of the GaLV-MS3-AR-bPa retrovirus. After centrifugation at 32° C. for 2 hours at 2000×g, the virus solution was removed, and the wells were washed with 1.5% HSA/PBS. After washing, to the wells were added 2×10⁶ peripheral blood mononuclear cells (PBMS) which had been separated from human peripheral blood by a Ficoll centrifugation method and then stimulated with OKT-3 and IL-2, and centrifuged at 32° C. for 10 minutes at 1000×g. After centrifugation, the plate was allowed to stand in a CO₂ incubator at 37° C. for 4 hours. The, cells were recovered, and seeded again on a new 24-well plate at a seeding density of 1×10⁶ cells/mL. On the next day, the similar procedure was performed to accomplish the second infection with the GaLV-MS3-AR-bPa retrovirus. Thereupon, after allowing to stand in a CO₂ incubator at 37° C. for 4 hours, the recovered cells were seeded again on a new 6-well plate at a seeding density of 2×10⁵ cells/mL.

### (4) Tetramer Assay

The amphotropic retrovirus-MS3-AR-bPa-infected SupT1 cell and a non-infected SupT1 cell as a negative control were reacted with 100 μg/mL of an HLA-A*0201/Aur-A₂₀₇₋₂₁₅ tetramer at 37° C. for 30 minutes after three days from infection, and the cells were then washed. The washed cells were subjected to flow cytometry analysis using FACS CantII (manufactured by BD).

As a result, a tetramer-positive rate was 0.2% in the case of the non-infected SupT1 cell as a negative control, while a tetramer-positive rate was 7.8% in the case of the MS-AR-bPa-introduced SupT1 cell. From the results, it was made clear that the gene products of the TCR α-chain and the TCR β-chain cloned from an #AUR-1 cell recognize a complex of Aur-A₂₀₇₋₂₁₅ and HLA-A*0201.

### (5) Cytotoxic Activity

T2 cells were washed with a RPMI1640 medium three times, and suspended in the RPMI1640 medium at a density of 1×10⁶ cells/mL. To 1 mL of the cell suspension was added Aur-A₂₀₇₋₂₁₅ at a final concentration of 20 μM, and the mixture was incubated at 37° C. for 16 hours. Similarly, 1 mL of a T2 cell suspension without Aur-A₂₀₇₋₂₁₅ was incubated at 37° C. for 16 hours. Both the cells were washed with a RPMI1640 medium containing no FCS three times, and 1×10⁶ cells were suspended in 1000 μL of a 10% FCS-containing RPMI1640 medium. To the cell suspension was added a 2.5 mM Calcein-AM (manufactured by Dojindo Laboratories) suspended in 10 μL of DMSO to label the cells at 37° C. for 1 hour. The resulting sample was used as a target cell for measurement of cytotoxic activity.

GaLV-MS3-AR-bPa retrovirus-infected PBMC and non-infected PBMC were suspended in a 10% FCS-containing RPMI1640 medium at a density of 3×10⁶ cells/mL, 1×10⁶ cells/mL, 3×10⁵ cells/mL or 1×10⁵ cells/mL (effector cells), and 100 μL of the suspension was placed into wells of a 96-well V-bottom plate. The target cells were suspended in a 10% FCS-containing RPMI1640 medium containing non-labeled K562 cells in a 20-fold amount of the target cells at a density of 1×10⁵ cells/mL, and 100 μL of the suspension was added to each well containing the effector cells. After reaction at 37° C. for 5 hours, the supernatant was recovered by centrifugation, and the fluorescent intensity of the supernatant was measured (ex: 485, em: 38 nm) with a fluorescent plate reader. From the measurement values of the fluorescent intensity, specific cytotoxic activity was calculated by the following equation:

Cytotoxicity (%)=[sample (florescent intensity)−spontaneous release (fluorescent intensity)]/[complete release (fluorescent intensity)−spontaneous release (fluorescent intensity)]×100 [Mathematical Expression 1]

In the equation, the spontaneous release corresponds to fluorescent intensity in a well containing no effector cell, and indicates the amount of spontaneous release from the target cell. The complete release indicates fluorescent intensity when Triton X-100 is added to the target cell, followed by destruction.

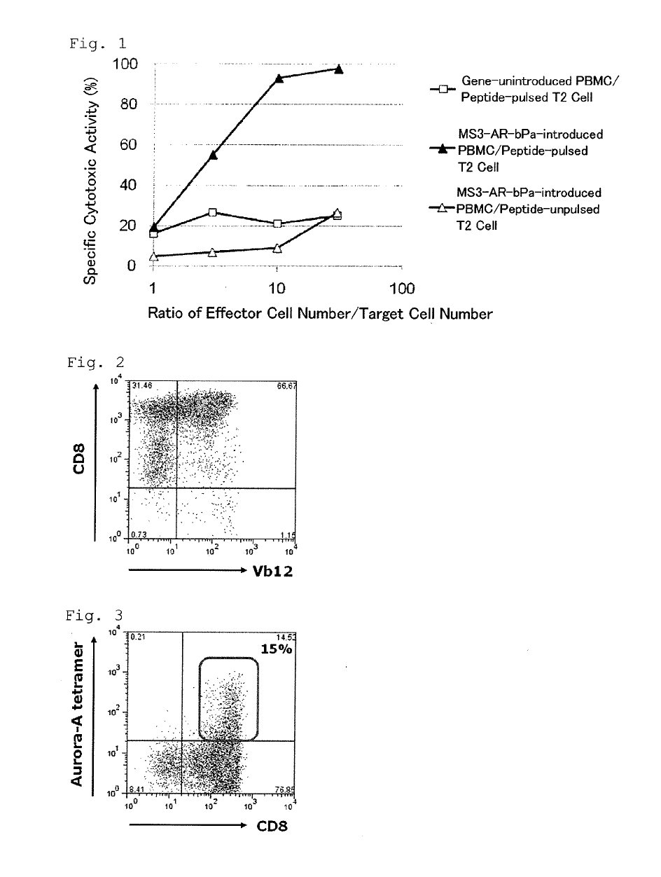

The results are shown in FIG. 1. The abscissa axis indicates the ratio of effector cell number/target cell number (E/T ratio) and the ordinate axis indicates specific cytotoxic activity (%). GaLV-MS3-AR-bPa retrovirus-infected PBMC exhibited cytotoxic activity on T2 cells which had been pulsed with Aur-A₂₀₇₋₂₁₅, and did not exhibit cytotoxic activity on T2 cells which had not been pulsed with Aur-A₂₀₇₋₂₁₅. In addition, non-infected PBMC did not exhibit cytotoxic activity on T2 cells which had been pulsed with Aur-A₂₀₇₋₂₁₅.

From the above results, it was made clear that genes encoding the TCR α-chain and the β-chain of an #AUR-1 cell impart Aur-A₂₀₇₋₂₁₅-specific and HLA-A*0201-restricted cytotoxic activity to a CTL clone and a peripheral blood-derived CD8 cell.

### Example 3

### (1) Introduction of Gene into Cell

GaLV-MS3-AR-bPa retrovirus-infected human CD8-positive lymphocytes were prepared by the same method as in Example 2 (3), and used as TCR gene-introduced human CD8-positive lymphocytes in the following experiment.

### (2) Flow Cytometry Analysis of TCR Gene-Introduced Human CD8-Positive Lymphocyte

After TCR gene-introduced human CD8-positive lymphocytes were reacted with an anti-human CD8 antibody (manufactured by Becton, Dickinson) and an anti-human Vb12 antibody (manufactured by Becton, Dickinson) which specifically recognizes a TCR β-chain V region, flow cytometry analysis was performed. The results are shown in FIG. 2. In FIG. 2, the X axis indicates a human Vb12 positive rate, and the Y axis indicates a human CD8-positive rate. As apparent from FIG. 2, 67% of the human CD8-positive lymphocytes in which the HLA-A*0201-restricted Aurora-A-specific TCR α/β gene of the present invention was introduced were human CD8-positive and human Vb12-positive.

### (3) Tetramer Assay

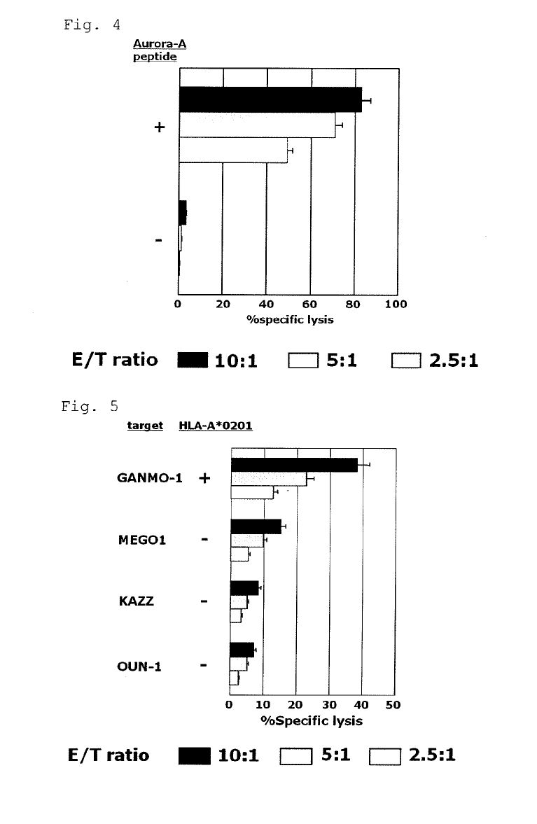

After the TCR gene-introduced human CD8-positive lymphocytes were reacted with an anti-human CD8 antibody and an HLA-A*0201/Aur-A₂₀₇₋₂₁₅ tetramer, flow cytometry analysis was performed. The results are shown in FIG. 3. In FIG. 3, the X axis indicates a human CD8-positive rate, and the Y axis indicates a tetramer-positive rate. As apparent from FIG. 3, 15% of the human CD8-positive lymphocytes in which the HLA-A*0201-restricted Aurora-A-specific TCR α/β gene of the present invention was introduced were human CD8-positive and HLA-A*0201/Aur-A₂₀₇₋₂₁₅ tetramer-positive. From the results of (2) and (3) of the present example, it was made clear that the vector encoding a TCR gene of the present invention shows an excellent introduction efficiency of a desired gene and, further, can impart tetramer-specific reactivity to a T cell.

### (4) Aurora-A-Specific Cytotoxic Activity of TCR Gene-Introduced Human CD8-Positive Lymphocyte

To 5×10³ cells of C1R-A2 which is an HLA-A*0201-positive B lymphocyte cell strain was added an Aur-A₂₀₇₋₂₁₅ peptide at a final concentration of 1 μM. Then, the cells were labeled with ⁵⁷Cr, and were used as target cells (T). Using TCR gene-introduced human CD8-positive lymphocytes as effector cells (E), the effector cells and the target cells were mixed at a ratio of E:T=10:1, 5:1 or 2.5:1, and then incubated at 37° C. for 4 hours. Similarly, using 5×10⁶ C1R-A2 cells without Aur-A₂₀₇₋₂₁₅ which were labeled with ⁵⁷Cr as target cells (T), the target cells were mixed with TCR gene-introduced human CD8-positive lymphocytes (E) at a ratio of E:T=10:1, 5:1, or 2.5:1, and then incubated at 37° C. for 4 hours. Separately, C1R-A2 cells with an added Aur-A₂₀₇₋₂₁₅ peptide or C1R-A2 cells without an Aur-A2₀₇₋₂₁₅ peptide were incubated at 37° C. for 4 hours without mixing with effector cells, and the cells were adopted as spontaneous release C1R-A2 cells. Further, the spontaneous release C1R-A2 cells were treated with 0.1% Triton X-100 (manufactured by Wako Pure Chemical Industries, Ltd.), and the cells were adopted as complete release C1R-A2 cells. ⁵⁷Cr released from the spontaneous release C1R-A2 cells, the complete release C1R-A2 cells, and the C1R-A2 cells mixed with effector cells at a variety of mixing ratios was measured with a γ counter. From the measurement values, specific cytotoxic activity was calculated by the following equation:

Cytotoxic activity (%)=[sample release ⁵⁷Cr (cmp)−spontaneous release ⁵⁷Cr (cmp)]/[complete release ⁵⁷Cr (cmp)−spontaneous release ⁵⁷Cr (cmp)]×100 [Mathematical Expression 2]

The analysis results of specific cytotoxic activity are shown in FIG. 4. In FIG. 4, the X axis indicates cytotoxic activity. As shown in FIG. 4, the human CD8-positive lymphocytes in which the HLA-A*0201-restricted Aurora-A-specific TCR α/β gene of the present invention was introduced exhibited high cytotoxic activity of 83%, 70.7% and 49.2% at E/T ratios of 10:1, 5:1 and 2.5:1, respectively. On the other hand, cytotoxic activity on the C1R-A2 cells without Aur-A₂₀₇₋₂₁₅ was not exhibited. From the above results, it was made clear that the TCR α/β gene of the present invention can impart Aur-A-specific cytotoxic activity to a T cell.

### Example 4

GaLV-MS3-AR-bPa retrovirus-infected human CD8-positive lymphocytes were prepared by the same method as in Example 2 (3), and used as effector cells. GANMO-1 which was an HLA-A*0201-positive and Aur-A-expressing leukemia cell strain; MEG01, KEZZ and OUN-1 which were HLA-A*0201-negative and Aur-A-expressing leukemia cell strains; HLA-A*0201-positive and Aur-A-non-expressing human PBMC; and human PHA-Blast prepared by adding 1 μg/ml of Phytohemagglutinin (PHA) to a human peripheral blood lymphocyte which was HLA-A*0201-positive and physiologically expressed Aur-A to some extent to bring in the normal division cycle were labeled with ⁵⁷Cr, and then used as target cells. Cytotoxic activity was calculated by the same method as in Example 3.

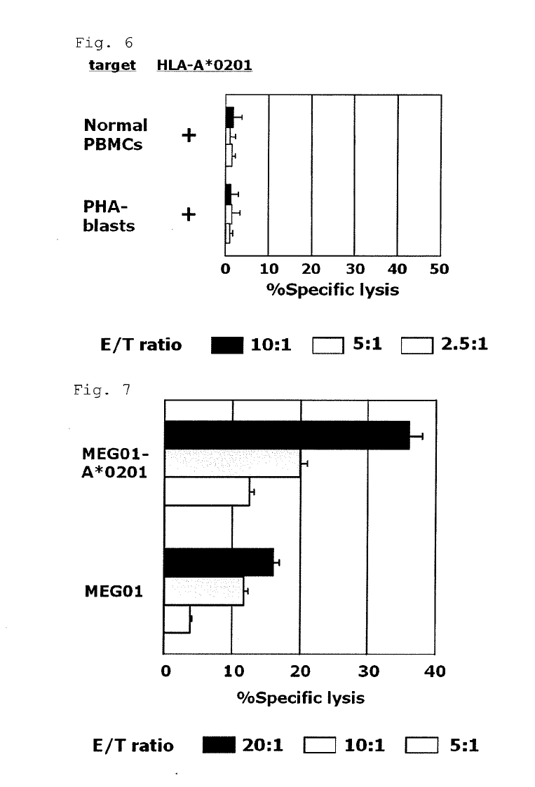

The analysis results of cytotoxic activity of the GaLV-MS3-AR-bPa retrovirus-infected human CD8-positive lymphocytes on leukemia cell strains are shown in FIG. 5. In FIG. 5, the X axis indicates cytotoxic activity. The human CD8-positive lymphocyte in which the HLA-A*0201-restricted Aurora-A-specific TCR α/β gene of the present invention was introduced exhibited effector cell number-dependent significant cytotoxic activity on the HLA-A*0201-positive and Aurora-A overexpressing leukemia cell strain GANMO-1. On the other hand, cytotoxic activity was not exhibited on the HLA-A*0201-negative and Aurora-A overexpressing leukemia cell strains MEG01, KAZZ and OUN-1. From the results, it was made clear that a T cell in which the TCR α/β gene of the present invention was introduced exhibits HLA-A*0201-restricted anti-leukemia cell activity.

The analysis results of cytotoxic activity of the GaLV-MS3-AR-bPa retrovirus-infected human CD8-positive lymphocytes on PBMC are shown in FIG. 6. In FIG. 6, the X axis exhibits cytotoxic activity. Unlike the case of leukemia cell strains, the GaLV-MS3-AR-bPa retrovirus-infected human CD8-positive lymphocytes did not exhibit cytotoxic activity on the human PBMC expressing little Aurora-A, and the normal division cycle-human PHA-Blast which physiologically expressed Aurora-A to some extent. From the results, it was shown that the human CD8-positive lymphocytes in which the HLA-A*0201-restricted Aurora-A-specific TCR α/β gene of the present invention is introduced HLA-restrictively recognizes and attacks only a tumor cell overexpressing the cancer antigen Aurora-A, and thus, its safety was confirmed.

### Example 5

GaLV-MS3-AR-bPa retrovirus-infected human CD8-positive lymphocytes were prepared by the same method as in Example 2 (3), and used as effector cells. An HLA-A*0201 gene was introduced into MEG01 which was an HLA-A*0201-negative and Aur-A-expressing leukemia cell strain to prepare an MEG01-A*0201 cell strain. The MEG01-A*0201 cell strain was further labeled with ⁵⁷Cr, and then used as a target cell. Cytotoxic activity was calculated by the same method as in Example 3. The E/T ratio was 20:1, 10:1, or 5:1.

The analysis results of cytotoxic activity are shown in FIG. 7. In FIG. 7, the X axis indicates cytotoxic activity. The MEG01 cell strain acquired sensitivity to the cytotoxic activity of the human CD8-positive lymphocyte in which the HLA-A*0201-restricted Aur-A-specific TCR α/β gene was introduced by the fact that the MEG01 cell strain became HLA-A*0201-positive. From the results, it was made clear that the human CD8-positive lymphocyte in which the HLA-A*0201-restricted Aurora-A-specific TCR-α/β gene of the present invention is introduced precisely recognizes a complex in which the target Aurora-A peptide derived from the cancer antigen Aurora-A protein in a leukemia cell is presented on HLA-A*0201, and exhibits anti-leukemia cell activity.

### INDUSTRIAL APPLICABILITY

According to the present invention, there are provided TCR α-chain and β-chain polypeptides derived from an HLA-A*0201-restricted CTL against Aur-A, and nucleic acids encoding the polypeptides. Since the nucleic acids can impart cytotoxic activity on a cell presenting an HLA-A*0201 molecule and an Aur-A₂₀₇₋₂₁₅ peptide to a T cell, they are useful in treating a cancer expressing Aur-A.

### SEQUENCE LISTING FREE TEXT

SEQ ID NO: 10; Oligo dT adaptor.
SEQ ID NO: 11; 5′-RACE primer.
SEQ ID NO: 12; Synthetic primer 3-TRalpha-C to amplify a DNA fragment encoding TCR alpha chain.
SEQ ID NO: 13; Synthetic primer 3-TRbeta-C1 to amplify a DNA fragment encoding TCR beta chain.
SEQ ID NO: 14; Synthetic primer 3-TRbeta-C2 to amplify a DNA fragment encoding TCR beta chain.
SEQ ID NO: 15-22; Synthetic primer.

1. A nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 2 of Sequence Listing, wherein the nucleic acid is selected from the group consisting of:
(1) a nucleic acid comprising a base sequence encoding a polypeptide consisting of the amino acid sequence set forth in SEQ ID NO: 5 of Sequence Listing,
(2) a nucleic acid comprising a base sequence encoding a polypeptide consisting of the amino acid sequence set forth in SEQ ID NO: 5 of Sequence Listing in which one to several amino acid residues are deleted, added, inserted or substituted,
(3) a nucleic acid comprising the base sequence set forth in SEQ ID NO: 6 of Sequence Listing, and
(4) a nucleic acid which can hybridize with a nucleic acid consisting of the base sequence set forth in SEQ ID NO: 6 of Sequence Listing or a complementary strand thereof under stringent conditions.

2. The nucleic acid according to claim 1, which encodes a polypeptide consisting of the amino acid sequence set forth in SEQ ID NO: 1 of Sequence Listing, or a polypeptide consisting of the amino acid sequence set forth in SEQ ID NO: 1 of Sequence Listing in which one to several amino acid residues are deleted, added, inserted or substituted.

3. A nucleic acid encoding a polypeptide which can compose an HLA-A*0201-restricted and Aur-A₂₀₇₋₂₁₅-specific T cell receptor together with a polypeptide consisting of the amino acid sequence set forth in SEQ ID NO: 1 of Sequence Listing, wherein the nucleic acid is selected from the group consisting of:
(1) a nucleic acid comprising a base sequence encoding a polypeptide consisting of the amino acid sequence set forth in SEQ ID NO: 7 of Sequence Listing,
(2) a nucleic acid comprising a base sequence encoding a polypeptide consisting of the amino acid sequence set forth in SEQ ID NO: 7 of Sequence Listing in which one to several amino acid residues are deleted, added, inserted or substituted,
(3) a nucleic acid comprising the base sequence set forth in SEQ ID NO: 8 of Sequence Listing, and
(4) a nucleic acid which can hybridize with a nucleic acid consisting of the base sequence set forth in SEQ ID NO: 8 of Sequence Listing or a complementary strand thereof under stringent conditions.

4. The nucleic acid according to claim 3, which encodes a polypeptide consisting of the amino acid sequence set forth in SEQ ID NO: 2 of Sequence Listing, or a polypeptide consisting of the amino acid sequence set forth in SEQ ID NO: 2 of Sequence Listing in which one to several amino acid residues are deleted, added, inserted or substituted.

5. A polypeptide encoded by the nucleic acid according to any one of claims 1 to 4.

6. An HLA-A*0201-restricted and Aur-A₂₀₇₋₂₁₅-specific T cell receptor, which is composed of a polypeptide encoded by the nucleic acid according to claim 1 or 2 and a polypeptide encoded by the nucleic acid according to claim 3 or 4.

7. A recombinant nucleic acid containing the nucleic acid according to any one of claims 1 to 4.

8. A vector in which at least one of the recombinant nucleic acids according to claim 7 is inserted.

9. A vector in which both a recombinant nucleic acid containing the nucleic acid according to claim 1 or 2 and a recombinant nucleic acid containing the nucleic acid according to claim 3 or 4 are inserted.

10. A combination of a vector in which a recombinant nucleic acid containing the nucleic acid according to claim 1 or 2 is inserted and a vector in which a recombinant nucleic acid containing the nucleic acid according to claim 3 or 4 is inserted.

11. A cell expressing an HLA-A*0201-restricted and Aur-A₂₀₇₋₂₁₅-specific T cell receptor, in which the vector according to claim 8 or 9 or the combination of vectors according to claim 10 is introduced.

12. The cell according to claim 11, which is a T cell or a cell which can differentiate into a T cell.

13. A carcinostatic agent containing the vector according to claim 9, or the combination of vectors according to claim 10, or the cell according to claim 11 or 12, as an active ingredient.

14. A method of treating a cancer including a step of administering the carcinostatic according to claim 13.

15. The vector according to claim 9, or the combination of the vectors according to claim 10, or the cell according to claim 11 or 12 for use in treatment of a cancer.
